(19) United States
(12) Patent Application Publication
(10) Pub. No.: US 2020/0085499 A1
(43) Pub. Date: Mar. 19, 2020

(54) HANDHELD DEVICE FOR TREATING AN ARTERY AND METHOD THEREOF

(71) Applicant: Ajoy I. Singh, Mumbai, Maharashtra (IN)

(72) Inventor: Ajoy I. Singh, Mumbai, Maharashtra (IN)

(21) Appl. No.: 15/750,864

(22) PCT Filed: Aug. 2, 2016
(86) PCT No.: PCT/IN2016/050258

§ 371 Date: Feb. 7, 2018

(30) Foreign Application Priority Data
Aug. 7, 2015 (IN) 2993/MUM/2015

(51) Int. Cl. A61B 18/22; A61B 90/00

(52) U.S. Cl. CPC A61B 18/22; A61B 2018/00577; A61B 90/06

(57) ABSTRACT

The present invention provides a handheld device and method for treating an artery having atherosclerosis. The handheld device for treating an artery comprises a handle for holding the device, an effector adapted to the handle for carrying out the treatment on the arteries, said effector having an ablating means, and a feedback controller for controlling process of treatment. Advantageously, the device allows for treating the artery without invasion of the blood vessel lumen, without damaging the tunica intima of the artery, and more importantly, the device can be used for treating early and mid-stage plaque formation in the arterial wall, which goes undetected in routine angiography.

### FIELD OF THE INVENTION

The present invention relates to a system for treating an artery, preferably, to a handheld device for treating an artery having atherosclerosis and a method of treating the arterythereof.

### BACKGROUND ART OF THE INVENTION

It is known that a normal artery (70) as shown in FIG. 1A has a lumen (60) defined by three layered structure, namely an innermost layer called as tunica intima (10), a middle layer called as tunica media (20) and anoutermost layer called as tunica adventitia (30). Atherosclerosis is a disorder of arteries that afflicts a large proportion of humanity. The disease is characterized by the blockages in arteries developed due to accumulation of cholesterol, fats and other chemicals between the tunica intima (10) and the tunica media (20) of the artery wall as shown in FIG. 1B. This accumulation is enveloped in a thin fibrous capsule (40). The fibrous capsule (40) with the accumulations of cholesterol, fats and other chemicals are often collectively referred to as [symbol] therosclerotic or atheromatous plaque [symbol] 50). This plaque (50) also contains cells of various types, predominantly macrophages, giant cells and smooth muscle cells. These cells are a consequence of the inflammatory nature of the disease process afflicts the wall of the artery. Thus, atherosclerosis is characterized by enlargement of the artery wall as shown in FIG. 1B. Principally restriction is due to atherosclerotic plaque bulging into the inner space (lumen) of the artery. This leads to increased arterial wall stiffness and reduced internal diameter of the artery, and consequently reduced blood flow through the artery.

The central portion of the atherosclerotic plaque is often semi-solid or liquid. This is referred to as [symbol] ecrotic core [symbol] in medical literature. Heart attack and brain strokes are caused if the contents of this necrotic core are released into the blood stream. The necrotic core contents when released into the blood stream trigger thrombus (Clot) formation, which prevents the flow of blood through the artery. This is how carotid and coronary thrombosis occurs.

Further, it has been recognized that atherosclerosis is a systemic disease. The blockages (stenosis) and a local manifestation of the disease process may also be widespread and distributed over a region of the arterial tree. The disease manifests itself in an angiogram as a local narrowing/blockage, but pathology studies confirm the existence of disease in areas that are not narrowed as well.

In real life, the biological process accompanying atherosclerosis is a lot more complex, including a self-healing mechanism of the human or animal body that attempts to minimize the constriction of the artery, called stenosis in medical terminology. The self-healing mechanism functions by “remodeling” the artery. The constituents of these prolonged depositions, called atheroma, include macrophage cells, cellular debris, dead cells and living cells, as well as the fibrous tissue covering of the atheroma itself. Over time, calcification can also occur between the atheroma layer and the underlying smooth muscle cell layer of the vessel wall.

Numerous medical equipments and techniques are available today for unblocking coronary arteries blocked by the atheromatous plaque. Chief among them are balloon angioplasty, Stents, rotational atherectomy, directional atherectomy and transluminal extraction atherectomy.

All above techniques approach the artery from inside. During an actual medical procedure, each of above techniques typically uses the catheter. For guidance, a guide wire is typically inserted first before the catheter. The catheter is then passed through the artery over the guide wire to reach the target area. The approach is through the artery lumen. All these procedures may cause some degree of injury to the innermost lining of the tunica intima. Hence, many of these procedures have a high rate of re-blockage due to cellular proliferation, which follows any injury to the tunica intima. The advent of drug eluting stents has reduced this incidence significantly, but limitations remain.

Treatment of atherosclerosis by all known present technologies is carried out or suggested to be carried out only when blockage of the vessel is more than 50% of internal diameter of the artery as the said technologies are nearly ineffective in treating early and about mid-stage plaque formation. This is particularly troublesome in view of the fact that mid-stage, vulnerable plaque formation with minimum lumen intrusion is now clinically considered to be even more dangerous owing to its tendency to rupture spontaneously, leading to immediate and severe heart attack or even instant death. It is estimated that two-thirds of all heart attacks are caused by thrombosis triggered by rupture of a plaque that was not bulging into the interior of the blood vessel. These plaques do not show up on routine angiography for the same reason (because they do not cause a narrowing of the artery). These plaques are detected by OCT or Coronary Computed Tomography angiography. Currently there is no effective means of treating these plaques and there is an urgent need to develop a therapeutic intervention addressing this preventable cause of heart and brain strokes.

The Patent number U.S. Pat. No. 6,669,686 granted to the present inventor discloses a method that avoids approaching atherosclerosis through the artery or from the inside of artery, more particularly, to a method for reducing the thickness of an arterial wall by ablation of the exterior of the artery wall by laser ablating or removing the exterior layer of the arterial wall the tunica intima and inner layers of the tunica media are protected from damage. The flexibility of the artery is improved due to the reduced effective wall thickness after ablation thus relieving stenosis and improving blood flow through the artery. However, the device used is complicated.

There is therefore a need of a means to solve at least one of the problems as discussed above for eliminating the said disease.

### SUMMARY OF THE INVENTION

Accordingly, the present invention provides in the first aspect provides a handheld device for treating an artery comprising a handle for holding the device, an effector adapted to the handle for carrying out the treatment on the arteries, said effector having an ablating means, and a feedback controller for controlling the process of treatment.

According to an embodiment of the present invention, a motor is adapted within and between the handle and the effector for rotating the effector.

In another embodiment of the present invention, the effector comprises,alone or in combination, at least one imaging means for carrying out imaging in real time, at least on pressure sensor and at least one gyroscope sensor connected to the feedback controller.

The present invention in second aspect provides a method for treating the artery using the handheld device of the first aspect by ablating the artery wall including the ablation of tunica adventitia and tunica media of the artery and the fibrous capsule of the atheromatous plaque for exposing the plaque to the natural defense system of the body, thereby eliminating atherosclerosis.

### BRIEF DESCRIPTION OF THE DRAWINGS

A preferred embodiment of the present invention will now be described with reference to the accompanying drawings (which in no way restrict the scope of the invention and are for the purpose of illustration only) in which:

FIG. 1A illustrates a normal artery;

FIG. 1B illustrates a diseased artery having a thickened wall due to atherosclerosis;

FIG. 2 illustrates a schematic view of a handheld device according to the present invention; and

FIG. 3 shows an effector cutting a diseased artery;

### DETAILED DESCRIPTION OF THE INVENTION

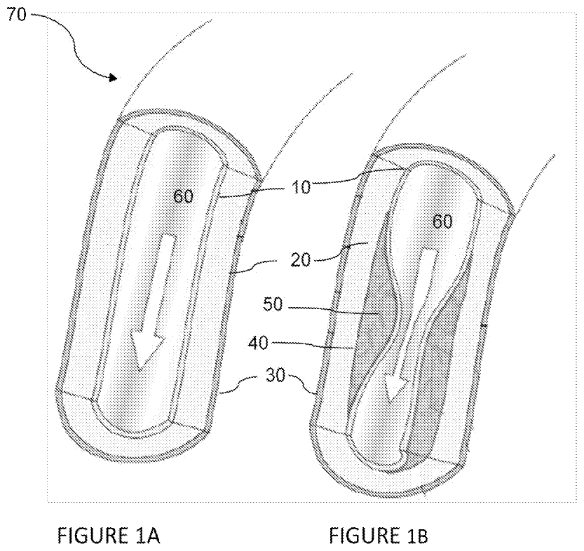

Referring to FIG. 1A, the artery 70 has a lumen 60. The artery wall comprises of tunica intima 10, tunica media 20 and tunica adventitia 30. Atherosclerosisin arteries is characterized by enlargement of the artery wall comprising tunica intima, tunica adventia and tunica media. Further, in FIG. 1B, the enlargement of artery wall has resulted in the restriction of the artery lumen. Principally the restriction is due to atherosclerotic plaque and/or enlarged tunica media. The atherosclerotic plaque 50 is contained in a fibrous capsule 40.

Whilst the following description concerns the ablation of an atherosclerosis artery, it will be appreciated that the present invention can be used to treat arterial walls thickened due to other causes such as cellular hyperplasia.

In general, the present invention provides a handheld device for treating an artery particularly atherosclerosis in the artery. The said handheld device comprises a handle for holding the device, an effector adapted to the handle for carrying out the treatment on the arteries and a feedback controller for controlling the process of treatment. According to the present invention, the effector is rotatably adapted to the handle of the device. According to the present invention, the effector comprises an ablating means and at least one pressure sensor to sense pressure on the artery in real time while cutting or ablating the artery.

According to a preferable embodiment, the effector comprises, one or in combination, at least one imaging means, at least one pressure sensor, and at least one gyroscope sensor connected to the feedback controller.

According to a preferable embodiment of the present invention, the handheld device comprises a motor adapted within and between the handle and the effector to rotate of the effector. Advantageously, the motor engages the effector with the handle effectively within 30° to the vertical, allowing to carry out ablation in the artery based on the pressure on the artery and to rotate of the effector based on the pressure. The preferable pressure on the artery is between 40 and 60 mm Hg. Advantageously, the motor folds the effector stopping the ablating process of the artery when the pressure exceeds 60 mm Hg.

According another preferable embodiment of the present invention, the effector also comprises an imaging means for carrying out imaging in real time.

According to the present invention, the ablating means is a fiber emitting high intensity laser for ablating the artery. According to the present invention, the laser is a femto-second laser. Alternatively, the ablation means can be a mechanical ablation device including at least one blade with pressure sensors, ultrasonic or any other known means.

According to the present invention, the imaging means is an Optical Coherence Tomography (OCT), ultrasonography, photo acoustic tomography or any other known means.

The present invention also provides a method for treating an artery, including the steps of approaching a diseased artery from the external side, placing the handheld device of the present invention over the diseased artery, and ablating artery wall including ablation of tunica adventitia and tunica media of the artery and a fibrous capsule of the atherosclerosis for exposing atheromatous plaque of the atherosclerosis to the natural defense system of the body thereby eliminating atherosclerosis. This partial thickness incision into the artery wall and plaque effectively conveys the semisolid and liquid contents of the [symbol] ecrotic core [symbol] to the outside of the vessel wall. This eliminates the possibility of rupture of vulnerable plaque and necrotic core into the artery and the triggering of thrombosis. Thus heart attack is prevented.

Advantageously, the method comprises a step of surveying thickness of the arterial wall and a length of the atherosclerosis in the artery prior to ablating the diseased artery with the help of imaging fibers provided in the handheld device and during ablating, calculating depth of ablating incision required to expose the atheromatous plaque from the diseased artery in real time.

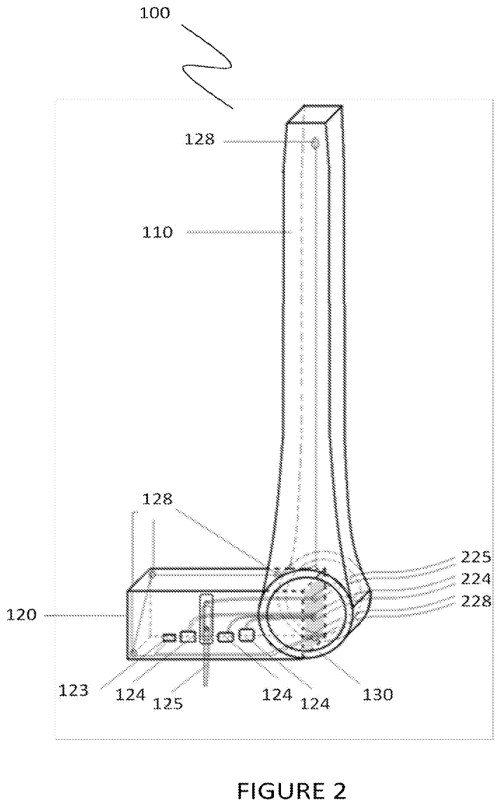

Referring FIG. 2 shows a handheld device (100) for treating a diseased artery (70) a preferable embodiment of the present invention. The handheld device (100) as shown in FIG. 2 comprises a handle (110) for holding the device, an effector (120) for effecting ablation or cutting the diseased artery and a motor (130) adapted within and between the handle (110) and the effector (120) to control the pressure of the effector on the artery while treating the diseased artery.

As shown in FIG. 2, the effector (120) comprises one pressure sensor (123) to determine a pressure exerted on the diseased artery while cutting or ablating the artery, two imaging sensors (124), and a cutting tool (125) adapted between the imaging means (126).

The cutting tool (125) shown is a mechanical blade. The cutting tool can be a laser, ultrasonic or any other know means that can be used for cutting or ablating the artery. According to the inventor of the present invention, though in FIG. 2 shown a mechanical blade, a preferable ablation means/cutting tools is an ablation fiber transmitting high intensity Femto-second pulsed laser having pulses preferably between 10 to 750 fs duration.

A gyroscope sensor and/or accelerometer sensor (128) (herein after referred as a gyroscope)is adapted in the handle (110) of the handheld device. The gyroscope sensor (128) keeps track of the angle between the handle and the effector. Whenever the angle between them goes beyond 30 degrees from vertical, the gyroscope sensor (128) activates the motor which folds the effector away from the artery towards the handle effectively stopping/preventing ablation. Further, during ablation/active imaging the gyroscope sensor (128) provides a feedback impulse to the motor to provide torque to the effector to maintain 40 to 60 mmHg pressure on the artery surface, as detected/reported by the pressure sensor on the under-surface of the effector.

As shown in FIG. 2, fibers (224,225, 228) extend from the imaging sensors (124), ablating means/cutting tool (125), and gyroscope sensor (128) to a feedback controller (not shown).

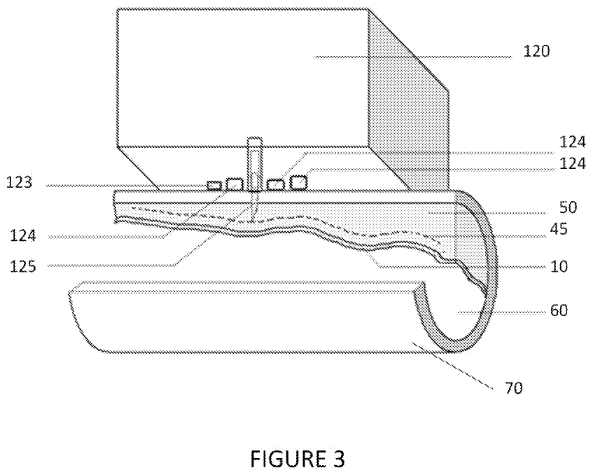

FIG. 3 shows the effector cutting a diseased artery along the ablation line 45 calculated by the feedback controller. According to the present invention, the operator approaches a diseased artery or artery to be treated from the external side and holds the handheld device of the present invention over the said artery with gentle pressure. Then the operator may survey the thickness of the arterial wall and a length of the atherosclerosis as well as depth to cut the artery for exposing the atherosclerosis in the artery prior to ablating the diseased artery with the help of imaging fibers provided in the handheld device of the present invention and triggers the ablation/cutting process by gently sliding the handheld device of the present invention along the artery surface to cover the length of the artery to be treated and ablates artery wall including ablation of tunica adventitia and tunica media of the artery and a fibrous capsule of the atherosclerosis for exposing atheromatous plaque of the atherosclerosis to the natural defense system of the body thereby eliminating atherosclerosis. This partial thickness incision into the artery wall and plaque effectively conveys the semisolid and liquid contents of the [symbol] ecrotic core [symbol] to the outside of the vessel wall. This eliminates the possibility of rupture of vulnerable plaque and necrotic core into the artery and the triggering of thrombosis. Thus heart attack is prevented. The cutting or ablation can be carried out by a laser or a mechanical blade. During ablating process, the imaging means continuously calculate a depth of ablating incision required to expose the atheromatous plaque from the diseased artery in real time, thereby controls the depth of cutting at various points and pressure sensor senses the pressure on the artery. If the pressure over the artery exceeds the upper limit, the motor in the handheld device folds the effector stopping the ablating process of the artery. According to the inventor of the present application, the pressure should not exceed 60 mm Hg.

The foregoing description of the invention has been set merely to illustrate the invention and is not intended to be limiting. Since modifications of the disclosed embodiments incorporating the substance of the invention may occur to a person skilled in the art, the invention should be construed to include everything within the scope of the disclosure.

1. A handheld device for treating an artery comprising;
a handle for holding the device,
an effector adapted to the handle for carrying out the treatment
on the arteries, said effector having an ablating means, and
a feedback controller for controlling process of treatment.

2. The device as claimed in the claim 1, wherein the effector is rotatably adapted to the handle of the device.

3. The device as claimed in claim 1, wherein a motor is adapted within and between the handle and the effector to rotate the effector.

4. The device as claimed in claim 1, wherein the effector further comprises at least one imaging means for carrying out imaging in real time, at least one pressure sensor, and at least one gyroscope sensor connected to the feedback controller.

5. The device as claimed in claim 1, wherein the motor engages the effector with the handle effectively within 30(10) to the vertical, allowing ablation to be carried out in the artery based on the pressure on the artery.

6. The device as claimed in claim 1, wherein the preferable pressure on the artery is between 40 and 60 mm Hg.

7. The device as claimed in claim 1 or 6, wherein the motor folds the effector stopping the ablating of the artery when the pressure exceeds 60 mm Hg.

8. The device as claimed in claim 1 or 4, wherein the effector preferably comprises two imaging means sensors and the ablating means adapted between the imaging means.

9. The device as claimed in claim 1, wherein the ablating means is a fiber emitting laser for ablating the artery.

10. The device as claimed in claim 9, wherein the fiber emits a femto-second pulsed laser.

11. The device as claimed in claim 10, wherein the femto-second pulsed laser is having pulses preferably between 10 to 750 fs duration.

12. The device as claimed in claim 1, wherein the ablation means can be a mechanical ablation device including at least one blade with pressure means, ultrasonic or any other known means.

13. The device as claimed in claim 4, wherein the imaging means is selected from an Optical Coherence Tomography (OCT), ultrasonography, photo acoustic tomography or any other known means.

14. A method of treating the artery using the handheld device of one of the preceding claims 1 to 13, the method comprising;
approaching a diseased artery from the external side,
placing the device over the diseased artery, and
ablating artery wall including ablation of tunica adventitia and tunica media of the artery and a fibrous capsule of the atherosclerosis for exposing atheromatous plaque of the atherosclerosis to the natural defense system of the body, thereby eliminating atherosclerosis.

15. The method as claimed in claim 14, wherein the method further comprises;
surveying thickness of the arterial wall and length of the atherosclerosis in the artery prior to ablating the diseased artery with the imaging means of the handheld device, and
calculating the depth of the ablating incision required to expose the atheromatous plaque from the diseased artery in real time.

16. The method as claimed in claim 14, wherein the step of ablation is carried out in the artery based on the pressure on the artery, wherein the preferable pressure on the artery is between 40 and 60 mm Hg.

17. The method as claimed in claim 14, wherein the step of ablation further comprises folding the effector and stopping the ablating of the artery when the pressure exceeds 60 mm Hg.
